(19) United States
(12) Patent Application Publication
(10) Pub. No.: US 2025/0228446 A1
(43) Pub. Date: Jul. 17, 2025

(54) METHOD OF MANUFACTURING OPTICAL FIBER BUNDLE CANNULA, AND MULTI-CHANNEL FIBER PHOTOMETRY SYSTEM

(71) Applicant: UNIVERSITY OF SCIENCE AND TECHNOLOGY OF CHINA, Hefei, Anhui (CN)

(72) Inventors: Guoqiang BI, Hefei, Anhui (CN); Keming ZHANG, Hefei, Anhui (CN); Qingyuan ZHU, Hefei, Anhui (CN); Pak-Ming LAU, Hefei, Anhui (CN)

(21) Appl. No.: 18/703,180

(22) PCT Filed: Oct. 8, 2022
(86) PCT No.: PCT/CN2022/123828

§ 371 Date: Apr. 19, 2024

(30) Foreign Application Priority Data
Oct. 22, 2021 (CN) 202111232249.1
Nov. 18, 2021 (WO) PCT/CN2021/131429

(51) Int. Cl. A61B 1/07; A61B 1/00; A61B 1/04; A61B 1/06; A61B 5/00

(52) U.S. Cl. CPC A61B 1/07; A61B 1/0011; A61B 1/043; A61B 1/0653; A61B 5/4064; A61B 2503/40

(57) ABSTRACT

A method of manufacturing an optical fiber bundle cannula and a multi-channel fiber photometry system based on the optical fiber bundle cannula are provided. The method includes: manufacturing an optical fiber positioning mold and forming positioning holes on the mold; inserting first ends of optical fibers into the positioning holes respectively; fixing exposed portions of the optical fibers close to the mold to the mold using a curing material so that a relative position of ends of the optical fibers inserted into the mold remains unchanged; inserting second ends of the optical fibers into a tubular portion so that the optical fibers located between the tubular portion and the mold form an umbrella-shaped portion; fixing the umbrella-shaped portion and part of the tubular portion using a curing material, so as to form a fixing portion; and extracting the optical fibers from the mold.

### CROSS REFERENCE TO RELATED APPLICATIONS

This application is a Section 371 National Stage Application of International Application No. PCT/CN2022/123828, filed on Oct. 8, 2022, entitled “METHOD OF MANUFACTURING OPTICAL FIBER BUNDLE CANNULA, AND MULTI-CHANNEL FIBER PHOTOMETRY SYSTEM”, which claims priority to the Chinese Patent Application No. 202111232249.1 filed on Oct. 22, 2021 and the International Application No. PCT/CN2021/131429 filed on Nov. 18, 2021, which are incorporated herein by reference in their entirety.

### TECHNICAL FIELD

The present disclosure relates to a field of neural signal recording, and in particular to a method of manufacturing an optical fiber bundle cannula used for neural signal recording, and a multi-channel fiber photometry system.

### BACKGROUND

A brain is an advanced center that controls behaviors and minds, and contains a large number of neurons and other types of cells. Recording and analyzing neural activities of a brain plays a crucial role in understanding various behaviors and mental states, diagnosing and treating mental illnesses, and developing artificial intelligence. At present, fiber photometry systems have been developed to observe neural activities of a brain.

### SUMMARY

The present disclosure provides a method of manufacturing an optical fiber bundle cannula, including: manufacturing an optical fiber positioning mold and forming a plurality of positioning holes on the optical fiber positioning mold; inserting first ends of a plurality of optical fibers into the positioning holes of the optical fiber positioning mold respectively; fixing exposed portions of the plurality of optical fibers close to the optical fiber positioning mold to the optical fiber positioning mold by using a curing material so that the relative position of ends of the plurality of optical fibers inserted into the optical fiber positioning mold remains unchanged; inserting second ends of the plurality of optical fibers into a tubular portion so that the optical fibers located between the tubular portion and the optical fiber positioning mold form an umbrella-shaped portion; fixing the umbrella-shaped portion and part of the tubular portion by using a curing material, so as to form a fixing portion; and extracting the optical fibers inserted into the optical fiber positioning mold from the optical fiber positioning mold.

In an example, the inserting first ends of a plurality of optical fibers into the positioning holes of the optical fiber positioning mold respectively includes: manufacturing an optical fiber alignment plate, where the optical fiber alignment plate is provided with a plurality of alignment holes configured for the optical fibers to pass through, where the alignment holes are consistent with the positioning holes on the optical fiber positioning mold in terms of position and number; placing the optical fiber alignment plate in parallel above the optical fiber positioning mold so that each of the alignment holes corresponds to a position of one of the positioning holes in a direction of an orthographic projection of the optical fiber positioning mold; passing the first ends of the plurality of optical fibers respectively through corresponding alignment holes, so as to pre-position the optical fibers; and inserting the first end of each optical fiber into the positioning hole; where the alignment hole is a through hole.

In an example, the method further includes: after fixing the exposed portions of the plurality of optical fibers close to the optical fiber positioning mold to the optical fiber positioning mold by using the curing material so that the relative position of the ends of the plurality of optical fibers inserted into the optical fiber positioning mold remains unchanged, removing the optical fiber alignment plate.

In an example, the positioning hole formed on the optical fiber positioning mold includes a blind hole or a variable diameter hole.

In an example, the method further includes: after inserting the second ends of the plurality of optical fibers into the tubular portion, inserting one or more reference optical fibers among the plurality of optical fibers so that the plurality of optical fibers are closely arranged around the reference optical fiber in a pattern of concentric circle, square, rectangle or other shapes, and one end of the reference optical fiber is close to the optical fiber positioning mold.

In an example, the curing material has a light shielding property.

In an example, the method further includes: after extracting the optical fibers inserted into the optical fiber positioning mold from the optical fiber positioning mold, cutting off portions of the plurality of optical fibers exposed from the tubular portion.

The present disclosure further provides a multi-channel fiber photometry system, including: at least one optical fiber bundle cannula manufactured by the above-mentioned method of manufacturing the optical fiber bundle cannula, where optical fibers of the optical fiber bundle cannula exposed from the fixing portion are inserted into targets to be measured so as to conduct excitation light to the targets to be measured and collect emission light which is generated by the targets to be measured upon the targets to be measured are excited; and an optical fiber detection device attached to the optical fiber bundle cannula, and configured to generate the excitation light, receive the emission light, and convert the optical signal into the electrical signal.

In an example, the optical fiber detection device includes: an optical fiber connector, where a tubular portion of the optical fiber bundle cannula is partially inserted into one end of the optical fiber connector to achieve an optical coupling between the optical fiber connector and the plurality of optical fibers of the optical fiber bundle cannula; an optical transceiver configured to generate the excitation light and receive the emission light; and an image sensor configured to generate an electrical signal representing an image of the an object to be imaged, according to the emission light received by the optical transceiver.

In an example, the optical transceiver includes: a housing having an interface optically coupled to the optical fiber connector; a light source provided in the housing and configured to generate a light beam; a first optical filter provided in the housing and configured to filter the light beam from the light source to generate the excitation light; and an optical conversion assembly provided in the housing and configured to guide the excitation light from the first optical filter to the optical fiber bundle cannula and convert the emission light from the optical fiber bundle cannula to the image sensor.

In an example, the optical conversion assembly includes: a dichroic mirror; an objective lens provided between the dichroic mirror and the optical fiber connector, where the objective lens is configured to receive the excitation light from the first optical filter reflected by the dichroic mirror and the emission light from the optical fiber bundle cannula, and further inject the excitation light onto the optical fiber bundle cannula; an eyepiece configured to receive the emission light which comes from the objective lens and is transmitted by the dichroic mirror; and a second optical filter configured to filter the emission light from the eyepiece and guide filtered emission light to the image sensor.

In an example, the optical fiber bundle cannula includes: a plurality of optical fibers; the tubular portion, where second ends of the plurality of optical fibers are held in the tubular portion; and a fixing portion, where middle portions of the optical fibers are fixed in the fixing portion, and the first ends of the optical fibers extend from the fixing portion in order to be inserted into the targets to be measured.

In an example, the multi-channel fiber photometry system further includes: a signal acquisition device configured to receive the electrical signal from the optical fiber detection device; and a commutating device coupled between the optical fiber detection device and the signal acquisition device to avoid a cable entanglement caused by a movement of an animal that contains the targets to be measured.

According to the method of manufacturing the optical fiber bundle cannula and the multi-channel fiber photometry system of the above embodiments of the present disclosure, the manufactured optical fiber bundle cannula has a high integration, a small size and a light weight, and the relative position of the plurality of optical fibers in the optical fiber bundle cannula may be set freely, so that a high flexibility is achieved to simultaneously record different brain regions.

### BRIEF DESCRIPTION OF THE DRAWINGS

FIG. 1 shows a flowchart of a method of manufacturing an optical fiber bundle cannula according to an embodiment of the present disclosure;

FIG. 2 shows a schematic diagram of a multi-channel fiber photometry system according to an embodiment of the present disclosure;

FIG. 3 shows a schematic diagram of an application scenario of a multi-channel fiber photometry system according to an embodiment of the present disclosure;

FIG. 4 shows a schematic diagram of another application scenario of a multi-channel fiber photometry system according to an embodiment of the present disclosure;

FIG. 5 shows a schematic diagram of an operation process of a method of manufacturing an optical fiber bundle cannula according to an embodiment of the present disclosure; and

FIG. 6 shows a comparison diagram between an angle deviation of optical fibers in a case that the optical fibers are pre-positioned by an alignment plate in the schematic diagram of the operation process shown in FIG. 5 and an angle deviation of optical fibers in a case that the optical fibers are not pre-positioned.

### DESCRIPTION OF REFERENCE NUMERALS IN THE ACCOMPANYING DRAWINGS

1—optical fiber connector; 2—tubular portion; 3—fixing portion; 4—optical fiber; 5—light source; 6—condenser lens; 7—first optical filter; 8—dichroic mirror; 9—objective lens; 10—eyepiece; 11—second optical filter; 12—image sensor; 13—housing; 14—optical fiber bundle cannula; 15—optical transceiver; 16—first commutating device; 17—signal acquisition device; 18—second commutating device; 19—optical fiber positioning mold; and 20—optical fiber alignment plate.

### DETAILED DESCRIPTION OF EMBODIMENTS

Embodiments of the present disclosure will be described below with reference to the accompanying drawings. It should be understood, however, that these descriptions are just exemplary and are not intended to limit the scope of the present disclosure. In the following detailed description, for ease of interpretation, many specific details are set forth to provide a comprehensive understanding of embodiments of the present disclosure. However, it is clear that one or more embodiments may also be implemented without these specific details. In addition, in the following description, descriptions of well-known structures and technologies are omitted to avoid unnecessarily obscuring the concepts of the present disclosure.

Terms used herein are just for the purpose of describing specific embodiments and are not intended to limit the present disclosure. The terms “including”, “containing”, etc. used herein indicate the presence of the feature, step, operation and/or component, but do not exclude the presence or addition of one or more other features, steps, operations or components.

All terms used herein (including technical and scientific terms) have the meanings generally understood by those skilled in the art, unless otherwise defined. It should be noted that the terms used herein shall be interpreted to have consistent meanings with the context of this specification, and shall not be interpreted in an idealized or overly rigid manner.

In a case of using the expression similar to “at least one of A, B or C”, it should be explained according to the meaning of the expression generally understood by those skilled in the art (for example, “a system including at least one of A, B or C” should include but not be limited to a system including A alone, a system including B alone, a system including C alone, a system including A and B, a system including A and C, a system including B and C, and/or a system including A, B and C).

In a fiber photometry system that has been developed, an optical fiber patch cord is used to connect one end to an animal brain and the other end to a recording device to collect neural activity signals from a single brain region. Such fiber photometry system may only record activities in a single brain region and may not record signals from a plurality of brain regions simultaneously. The brain is generally active in a plurality of brain regions simultaneously, and a lot of information may be lost in the case of recording from a single brain region. In addition, such fiber photometry system has a large-volume fiber optic signal acquisition device, with a three-dimensional size generally ranging from a dozen centimeters to tens of centimeters, and a weight generally ranging from several kilograms to a dozen kilograms. In practical applications, a device with such size and weight may only be placed on a shelf and connected to an animal head through an optical fiber patch cord. In such fiber photometry system, it is difficult to record for a long time because the optical fiber patch cord may become entangled with a movement of the animal. It is also difficult to record complex and energetic behaviors, because firstly, the optical fiber patch cord has a certain rigidity and weight and may hinder the movement of the animal, and secondly, a deformation of the optical fiber patch cord caused by the energetic activity of the animal may also cause a signal distortion.

In a multi-channel fiber photometry system that has been developed, a one-to-multiple optical fiber bundle patch cord is used to record activities in a plurality of brain regions. Several separate ends of the optical fiber bundle patch cord are connected to different brain regions of the animal, and an integrated end is connected to a fiber optic acquisition device. Although the multi-channel fiber photometry system may record from a plurality of brain regions, it has a large volume and a heavy weight, and the fiber entanglement and deformation may cause the signal distortion. Moreover, the solution of fiber bundle patch cord increases the burden on the animal head, and a load of the plurality of optical fiber patch cords simultaneously connected to the brain severely affects its use in small animals.

In a highly-integrated multi-channel fiber photometry system that has been developed, commercial row connectors are employed, a plurality of optical fibers are implemented as an optical fiber bundle cannula implanted in an animal brain, and a lightweight optical fiber patch cord is used to transmit activity signals from a plurality of brain regions to an acquisition device. Although a high-density fiber bundle cannula is proposed in such multi-channel fiber photometry system, the connector with fixed hole positions limits a flexible distribution of fiber ends, and the fiber ends may only be distributed at a fixed multiple spacing. Moreover, a back-end acquisition device has a large size, and the entanglement of the optical fiber patch cord and the fiber deformation in a case of long-time recording may cause the signal distortion.

The present disclosure provides a method of manufacturing an optical fiber bundle cannula and a multi-channel fiber photometry system, so as to improve a flexibility of the fiber photometry system for simultaneously recording from different brain regions.

According to a general inventive concept of an aspect of the present disclosure, a method of manufacturing an optical fiber bundle cannula is provided, including operations S100 to S600.

In some embodiments of the present disclosure, in operation S100, an optical fiber positioning mold is manufactured, and a plurality of positioning holes are formed on the optical fiber positioning mold.

In some embodiments of the present disclosure, in operation S200, first ends of a plurality of optical fibers are inserted into the positioning holes of the optical fiber positioning mold respectively.

In some embodiments of the present disclosure, in operation S300, exposed portions of the plurality of optical fibers close to the optical fiber positioning mold are fixed to the optical fiber positioning mold by using a curing material, so that the relative position of ends of the plurality of optical fibers inserted into the optical fiber positioning mold remains unchanged.

In some embodiments of the present disclosure, in operation S400, second ends of the plurality of optical fibers are inserted into a tubular portion so that the optical fibers located between the tubular portion and the optical fiber positioning mold form an umbrella-shaped portion.

In some embodiments of the present disclosure, in operation S500, the umbrella-shaped portion and part of the tubular portion are fixed using a curing material, so as to form a fixing portion.

In some embodiments of the present disclosure, in operation S600, the optical fibers inserted into the optical fiber positioning mold are extracted from the optical fiber positioning mold.

In some embodiments of the present disclosure, operation S200 includes steps S210 to S240.

In step S210, an optical fiber alignment plate is manufactured. The optical fiber alignment plate is provided with a plurality of alignment holes configured for the optical fibers to pass through, and the alignment holes are consistent with the positioning holes on the optical fiber positioning mold in terms of position and number.

In step S220, the optical fiber alignment plate is placed in parallel above the optical fiber positioning mold, so that each of the alignment holes corresponds to a position of one of the positioning holes in a direction of an orthographic projection of the optical fiber positioning mold.

In step S230, the first ends of the plurality of optical fibers are passed through the corresponding alignment holes respectively, so as to pre-position the optical fibers.

In step S240, the first end of each optical fiber is inserted into the positioning hole.

In some embodiments of the present disclosure, operation S300 further includes removing the optical fiber alignment plate, after fixing the exposed portions of the plurality of optical fibers close to the optical fiber positioning mold to the optical fiber positioning mold by using the curing material so that the relative position of the ends of the plurality of optical fibers inserted into the optical fiber positioning mold remains unchanged.

In some embodiments of the present disclosure, the positioning holes formed on the optical fiber positioning mold include but are not limited to blind holes or variable diameter holes.

In an exemplary embodiment, at least two of the plurality of blind holes formed on the optical fiber positioning mold have different depths.

Specifically, all the plurality of blind holes formed on the optical fiber positioning mold have different depths, in order to meet requirements of a length of the optical fiber extending from the fixing portion.

In another exemplary embodiment, a plurality of variable diameter holes are formed on the optical fiber positioning mold.

Specifically, each variable diameter hole has a hole diameter that decreases in a depth direction from an opening position.

Furthermore, the hole diameter at the opening position of the variable diameter hole is greater than or equal to a diameter of the optical fiber to allow the optical fiber to pass through, and a hole diameter of an interior of the variable diameter hole is less than the diameter of the optical fiber to limit an appropriate depth of the optical fiber extending into the optical fiber positioning mold.

According to a general inventive concept of another aspect of the present disclosure, a multi-channel fiber photometry system is provided, including: at least one optical fiber bundle cannula manufactured by the above-mentioned method of manufacturing the optical fiber bundle cannula, where the optical fibers of the optical fiber bundle cannula exposed from the fixing portion are inserted into the targets to be measured so as to transmit excitation light to the targets to be measured and collect emission light generated by the targets to be measured upon the targets to be measured are excited; and an optical fiber detection device attached to the optical fiber bundle cannula, where the optical fiber detection device is used to generate the excitation light, receive the emission light, and convert the optical signal into the electrical signal.

According to the method of manufacturing the optical fiber bundle cannula and the multi-channel fiber photometry system of the embodiments of the present disclosure, a spatial position between the plurality of optical fibers may be freely set for the targets to be measured, so that the flexibility of simultaneously recording different targets to be measured may be improved.

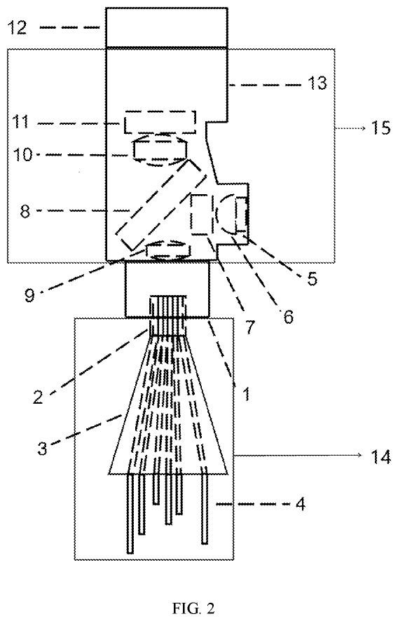

FIG. 2 shows a schematic diagram of a multi-channel fiber photometry system according to an embodiment of the present disclosure.

As shown in FIG. 2, the multi-channel fiber photometry system of the embodiment of the present disclosure includes an optical fiber bundle cannula 14 and an optical fiber detection device attached to the optical fiber bundle cannula 14. The optical fiber bundle cannula 14 is used to transmit excitation light to the targets to be measured and collect emission light generated by the targets to be measured upon the targets to be measured are excited. The optical fiber detection device is used to generate the excitation light, receive the emission light, and convert the optical signal into the electrical signal.

In some embodiments of the present disclosure, the optical fiber bundle cannula 14 includes a plurality of optical fibers 4, a tubular portion 2, and a fixing portion 3. First ends (lower ends in FIG. 2) of the plurality of optical fibers 4 are inserted into the targets to be measured (such as the brain regions of a measured animal). Second ends (upper ends in FIG. 2) of the plurality of optical fibers 4 are held in the tubular portion 2. Middle portions of the optical fibers 4 are fixed in the fixing portion, and the first ends of the optical fibers 4 extend from the fixing portion 3 to be inserted into the target to be measured.

In some optional embodiments of the present disclosure, a spatial position between the first ends of the optical fibers 4 may be freely set for different targets to be measured.

In some embodiments of the present disclosure, the first ends of the optical fibers 4 extend from the fixing portion 3, in order to be inserted into the targets to be measured, for example, into different targeted brain regions of an animal head.

In some optional embodiments of the present disclosure, the plurality of optical fibers 4 may be plastic optical fibers or optical fibers made of other materials.

In some embodiments of the present disclosure, the fixing portion 3 is used to fix the middle portions of the plurality of optical fibers 4, so as to ensure that the relative spatial position of the first ends of the plurality of optical fibers 4 remains unchanged.

In some embodiments of the present disclosure, the fixing portion 3 is formed by curing the middle portions of the plurality of optical fibers 4 using a curing material.

In such embodiments, the curing material may be dental fluid resin, light-curing glue, epoxy resin, silicone rubber, PDMS, or agarose.

In some optional embodiments of the present disclosure, the curing material is a material having a light shielding property.

In some embodiments of the present disclosure, the plurality of optical fibers 4 are closely arranged. The tubular portion 2 concentrates and fixes the second ends of the plurality of optical fibers 4, and the second ends of the plurality of optical fibers 4 are held in the tubular portion.

In such embodiments, a tubular portion 2 with an appropriate inner diameter is selected according to the number and diameter of the plurality of optical fibers 4. The tubular portion 2 may be a tubular portion or other fixing structures.

In some embodiments of the present disclosure, the tubular portion 2 is a hard capillary tube. Optionally, for example, the tubular portion 2 is a stainless steel capillary tube.

In some embodiments of the present disclosure, the plurality of optical fibers 4 include six plastic optical fibers with a diameter of 250 microns and different lengths, which target six brain regions. The fixing portion 3 has a height of 7 mm, and the fixing portion 3 may be a cone or have other shapes. The tubular portion 2 has a height of 2 mm.

In some optional embodiments of the present disclosure, the optical fiber bundle cannula 14 further includes a reference optical fiber. Instead of being inserted into the measured target, the reference optical fiber is used to reflect the excitation light as a comparison with optical signals in the plurality of optical fibers 4.

In some optional embodiments of the present disclosure, one or more reference optical fibers may be provided.

In some optional embodiments of the present disclosure, the plurality of optical fibers 4 are closely arranged around the reference optical fiber in a pattern of concentric circle, square, rectangle or other shapes.

In an embodiment of the present disclosure, the reference optical fiber is a plastic optical fiber with a diameter of 250 microns, and the plurality of optical fibers 4 are closely arranged around the reference optical fiber in a pattern of concentric circle.

In such embodiments, an end face of the reference optical fiber at the same end as the first end of the optical fiber 4 is provided with a metallic paint coating to enhance an optical signal in the reference optical fiber.

The optical fiber detection device includes: an optical fiber connector 1, where the tubular portion 2 of the optical fiber bundle cannula is partially inserted into one end of the optical fiber connector to achieve an optical coupling between the fiber optic connector 1 and the plurality of optical fibers 4 of the optical fiber bundle cannula 14; an optical transceiver 15 used to generate the excitation light and receive the emission light; and an image sensor 12 used to generate an electrical signal representing an image of the object to be imaged, according to the emission light received by the optical transceiver 15.

In some optional embodiments of the present disclosure, the optical fiber detection device may have a weight of less than 2 grams.

In an embodiment of the present disclosure, the optical fiber detection device 15 has a dimension of 7×7×16 mm and a weight of 0.8 g.

In some optional embodiments of the present disclosure, the optical fiber connector 1 is a tubular connector, and the tubular portion 2 of the optical fiber bundle cannula 14 is partially inserted into one end of the optical fiber connector 1 to achieve the optical coupling between the optical fiber connector 1 and the plurality of optical fibers 4 in the optical fiber bundle cannula 14.

In an embodiment of the present disclosure, the optical fiber connector 1 is a tubular connector with an outer dimension of 3×3×3 mm. The optical fiber connector 1 has an M0.6 micro screw on each of opposite sides, which is used to fix to the optical transceiver 15.

In an embodiment of the present disclosure, the optical transceiver 15 includes: a housing 13 including an interface optically coupled to the optical fiber connector 1; a light source 5 provided in the housing 13 and used to generate a light beam; a first optical filter 7 provided in the housing 13, and used to filter the light beam from the light source 5 to generate the excitation light; and an optical conversion assembly provided in the housing 13, and used to guide the excitation light from the first optical filter 7 to the optical fiber bundle cannula 14 and guide the emission light from the optical fiber bundle cannula 14 to the image sensor 12.

In an embodiment of the present disclosure, the housing 13 includes an interface optically coupled to the optical fiber connector 1, and an inner surface of the housing 13 has an uneven structure to reduce stray light in the optical transceiver 15. The housing 13 has a light shielding degree of greater than 90%. In an embodiment of the present disclosure, the housing 13 may be made of a 3D printed black plastic or other light shielding materials.

In an embodiment of the present disclosure, the light source 5 is a micro LED with a dimension of 1.7×1.3×0.4 mm, and is provided on a lower right side of an inner wall of the housing 13. The micro LED emits a blue light beam with a dominant wavelength of 470 nanometers. It is also possible for the light source 5 to include two micro LEDs with different dominant wavelengths, which emit a dual-color light beam with two dominant wavelengths. It is also possible for the light source 5 to include a plurality of micro LEDs with different dominant wavelengths.

In an embodiment of the present disclosure, the first optical filter 7 is provided on a side of the light source 5 away from the inner wall of the housing 13, and has a length and a width not greater than 5 mm. In an embodiment of the present disclosure, the first optical filter 7 is a disc with a diameter of 2 mm, a thickness of 1 mm, a wavelength selection range of 460 to 480 nm, and an OD value of at least 6.

In some optional embodiments of the present disclosure, a condenser lens 6 may be further provided between the light source 5 and the first optical filter 7 to concentrate and collect the light beam generated by the light source 5. The condenser lens 6 has a diameter of not greater than 5 mm. In an embodiment of the present disclosure, the condenser lens 6 is a hemispheric lens with a diameter of 2 mm, and one end of a plane of the hemispheric lens is arranged closely to a left side of the light source 5. The first optical filter 7 is arranged vertically on a left side of the condenser lens 6 and is 0.1 mm away from a high point of the condenser lens 6.

The optical conversion assembly includes: a dichroic mirror 8; an objective lens 9 provided between the dichroic mirror 8 and the optical fiber connector 1, where the objective lens 9 is used to receive excitation light from the first optical filter 7 reflected by the dichroic mirror 8 and the emission light from the optical fiber bundle cannula 14, and further inject the excitation light onto the optical fiber bundle cannula 14; an eyepiece 10 used to receive the emission light which is generated from the objective lens 9 and is transmitted by the dichroic mirror 8; and a second optical filter 11 used to filter the emission light from the eyepiece 10 and guide the filtered emission light to the image sensor 12.

In an embodiment of the present disclosure, the optical conversion assembly is arranged on a side of the first optical filter 7 away from the light source 5. The dichroic mirror 8 is arranged adjacent to the first optical filter 7 with an upper end being tilted to the right. The objective lens 9 is arranged below the dichroic mirror 8, and the eyepiece 10 is arranged above the dichroic mirror 8. The second optical filter 11 is arranged above the eyepiece 10.

The dichroic mirror 8 is arranged on the other side of the first optical filter 7, with an upper end of a reflection and transmission surface being tilted to form a vertical angle of 45° with respect to the first optical filter 7. The dichroic mirror 8 has a width of not more than 5 mm, and is used to reflect the excitation light and transmit the emission light.

In an embodiment of the present disclosure, the dichroic mirror 8 has a length of 5.2 mm, a width of 3 mm, a thickness of 1.1 mm, and a center wavelength of 500 nanometers. The upper end of the reflection and transmission surface of the dichroic mirror 8 is tilted to the right to form a vertical angle of 45° with respect to the first optical filter 7. A center point of the dichroic mirror 8 is 1.8 mm away from the first optical filter 7.

In some optional embodiments of the present disclosure, the objective lens 9 is located below the dichroic mirror 8 and has a diameter of not more than 5 mm.

In an embodiment of the present disclosure, the objective lens 9 is a biconvex lens with a diameter of 2 mm and a focal length of 2 mm, which is placed vertically below the dichroic mirror 8. The highest point of an upper surface of the objective lens 9 is 1.6 mm away from the center point of the lower surface of the reflection and transmission surface of the dichroic mirror 8. The center point of the objective lens 9 coincides with the center point of the reflection and transmission surface of the dichroic mirror 8 in the vertical direction.

In some optional embodiments of the present disclosure, the eyepiece 10 is located above the dichroic mirror 8 and has a diameter of not more than 5 mm.

In an embodiment of the present disclosure, the eyepiece 10 is a biconvex lens with a diameter of 3 mm and a focal length of 6.3 mm, which is placed above the dichroic mirror 8.

In some optional embodiments of the present disclosure, the second optical filter 11 is located above the eyepiece 10, and has a length and a width of not greater than 5 mm.

In an embodiment of the present disclosure, the second optical filter 11 is a disc with a diameter of 3.5 mm, a thickness of 1 mm, a wavelength selection range of 515 to 535 nm, and an OD value of at least 6. The second optical filter 11 is placed above the eyepiece 10, and is 0.1 mm away from the upper surface of the eyepiece 10.

In some optional embodiments of the present disclosure, the image sensor 12 is provided outside the housing 13 at an end away from the optical fiber bundle cannula 14 and is used to generate an electrical signal representing an image of the object to be measured according to the emission light received by the optical transceiver 15.

In an embodiment of the present disclosure, the image sensor 12 is a 600-line analog CMOS with a dimension of 6.5×6.5×2 mm, which is located above the second optical filter 11 and is 3.4 mm away from the second optical filter 11.

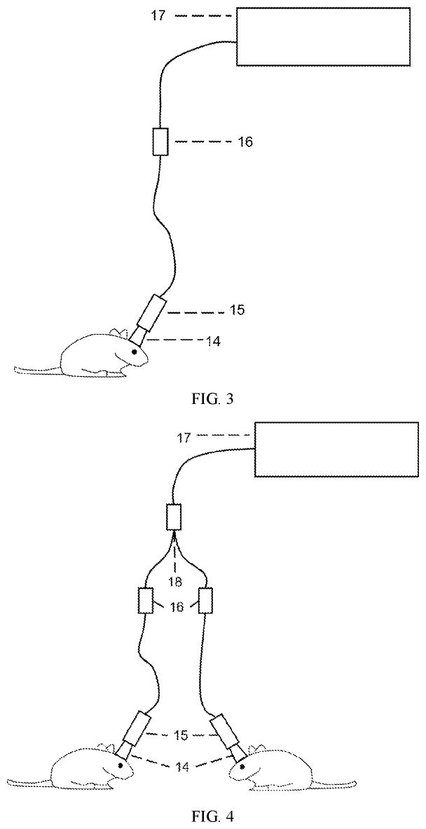

FIG. 3 shows a schematic diagram of an application scenario of a multi-channel fiber photometry system according to an embodiment of the present disclosure.

In order to record a signal of the target to be measured, for example, simultaneously record signals in a plurality of targeted brain regions, a fluorescent probe, such as a calcium activity indicator GCaMP6s is injected into the targeted brain regions in advance, and the first ends of the optical fibers 4 are inserted into the targeted brain regions. The light beam emitted by the light source 5 is condensed by the condenser lens 6 and then filtered and purified by the first optical filter 7 to form excitation light. The excitation light is reflected by the dichroic mirror 8 onto the objective lens 9 and is transmitted to the targeted brain regions by the optical fiber bundle cannula 14 to excite the calcium activity indicator GCaMP6s that has been injected into the targeted brain regions in advance. A green fluorescence signal emitted by GCaMP6s after excitation is collected as emission light by the first end of the optical fiber 4. The emission light passes through the fixing portion 3 and the tubular portion 2 and is received by the objective lens 9, then transmitted by the dichroic mirror 8 and received by the eyepiece 10, then filtered and purified by the second optical filter 11, and finally collected by the image sensor 12 at a frequency of 25 frames per second. The image sensor 12 may convert the optical signal of the emission light into an electrical signal.

As shown in FIG. 3, in an embodiment, the multi-channel fiber photometry system may further include a signal acquisition device 17. The signal acquisition device 17 is coupled to the optical fiber detection device and is configured to receive the electrical signal from the optical fiber detection device. In an embodiment of the present disclosure, the signal acquisition device 17 is a computer installed with an analog video acquisition card.

As shown in FIG. 3, the multi-channel fiber photometry system may further include a commutating device coupled between the optical fiber detection device and the acquisition device 17. The commutating device is used to avoid a cable entanglement caused by an animal movement. The commutating device may be a conductive slip ring or an active commutator. In an embodiment of the present disclosure, the commutating device is a first commutating device 16, which is a 4-channel conductive slip ring with a diameter of 6.5 mm and a length of 10 mm.

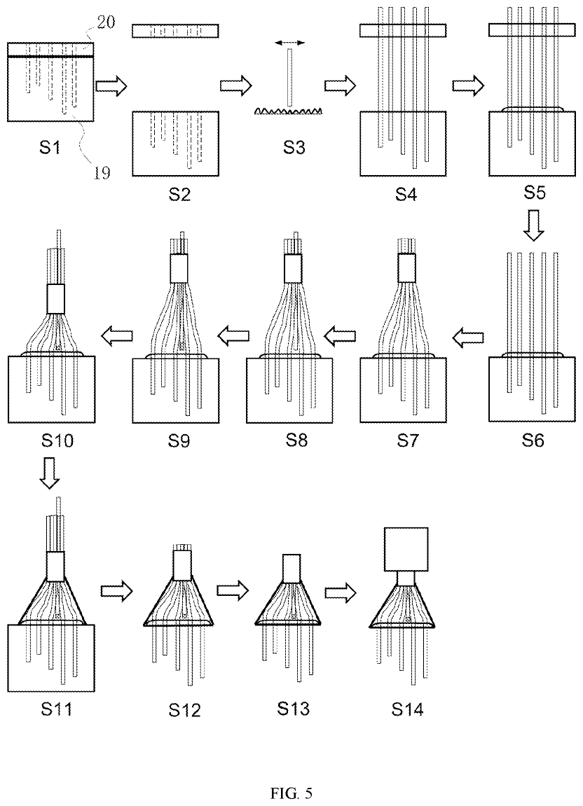

FIG. 4 shows a schematic diagram of another application scenario of a multi-channel fiber photometry system according to an embodiment of the present disclosure.

As shown in FIG. 4, in another embodiment of the present disclosure, two multi-channel fiber photometry systems according to the embodiments of the present disclosure are used to detect two measured brains. It is possible for the commutating device to include two first commutating devices 16 and a second commutating device 18. The second commutating device 18 is an 8-channel conductive slip ring with a diameter of 6.5 mm and a length of 10 mm.

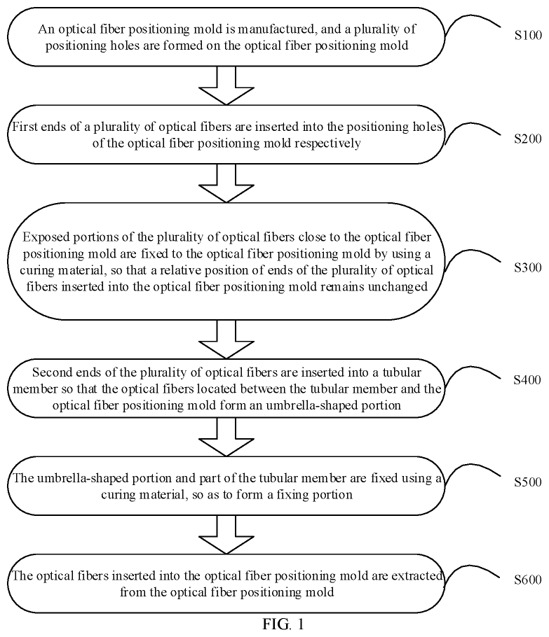

FIG. 1 shows a flowchart of a method of manufacturing an optical fiber bundle cannula according to an embodiment of the present disclosure.

As shown in FIG. 1 and FIG. 5, the present disclosure further provides a method of manufacturing an optical fiber bundle cannula 14, including operation S100 to operation S600.

In operation S100, an optical fiber positioning mold is manufactured, and a plurality of positioning holes are formed on the optical fiber positioning mold.

In operation S200, first ends of a plurality of optical fibers 4 are inserted into the positioning holes of the optical fiber positioning mold respectively.

In operation S300, exposed portions of the plurality of optical fibers 4 close to the optical fiber positioning mold are fixed to the optical fiber positioning mold by using a curing material, so that the relative position of ends of the plurality of optical fibers 4 inserted into the optical fiber positioning mold remains unchanged.

In operation S400, second ends of the plurality of optical fibers 4 are inserted into a tubular portion 2 so that the optical fibers 4 located between the tubular portion 2 and the optical fiber positioning mold form an umbrella-shaped portion.

In operation S500, the umbrella-shaped portion and part of the tubular portion are fixed using a curing material, so as to form a fixing portion 3.

In operation S600, the optical fibers inserted into the optical fiber positioning mold are extracted from the optical fiber positioning mold.

FIG. 5 shows a schematic diagram of an operation process of a method of manufacturing an optical fiber bundle cannula according to an embodiment of the present disclosure.

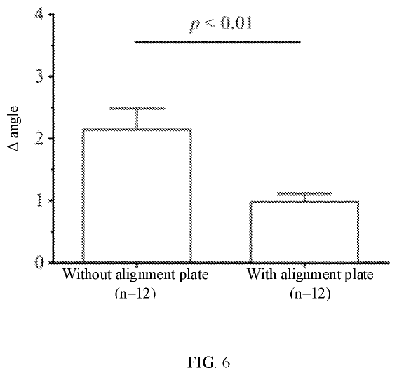

FIG. 6 shows a comparison diagram of an angle deviation of optical fibers in a case of pre-positioning the optical fiber using an alignment plate in the schematic diagram of the operation process shown in FIG. 5 and in a case of not pre-positioning the optical fiber.

As shown in FIG. 5, according to an embodiment of the present disclosure, the method of manufacturing the optical fiber bundle cannula 14 may specifically include steps S1 to S14.

In step S1, an optical fiber positioning mold is manufactured by drilling positioning holes of appropriate depth and diameter at corresponding positions of an animal skull model by using an engraving machine or a computer numerical control machine tool according to the number, size and three-dimensional coordinates of the targeted brain regions to be recorded. For example, the animal skull model may be a small hard block having the same size as the animal head, or a 1:1 skull model with a real skull structure produced by a 3D printing technology. In an embodiment of the present disclosure, the optical fiber positioning mold is manufactured by drilling six positioning holes with appropriate depth and a diameter of 250 micron at corresponding positions of a transparent acrylic block with a dimension of 20×20×10 mm.

In step S2, an optical fiber alignment plate 20 is manufactured according to the position and number of the positioning holes on the optical fiber positioning mold 19, so that a hole diameter of the optical fiber alignment plate is consistent with that of the positioning hole.

In step S3, the first ends of the plurality of optical fibers 4 are polished to be smooth without scratches. In an embodiment of the present disclosure, one ends of seven plastic optical fibers with a length of 30 mm and a diameter of 250 microns are polished using 1500-mesh, 3000-mesh, 7000-mesh, 10000-mesh and 12000-mesh sandpaper respectively to be smooth without scratches.

In step S4, the first ends of the plurality of optical fibers 4 are inserted into corresponding alignment holes provided on the optical fiber alignment plate 20, and the first ends are passed through the alignment holes.

In step S5, the first ends of the plurality of optical fibers 4 are inserted into the positioning holes until encountering a great resistance, and remaining portions are left outside the optical fiber positioning mold. In an embodiment of the present disclosure, six of the seven optical fibers are inserted into the positioning holes of the optical fiber positioning mold.

In step S6, a curing material is applied to the exposed portions of the plurality of optical fibers 4 close to the optical fiber positioning mold. In an embodiment of the present disclosure, a black light-curing dental resin is applied to a region where a surface of the optical fiber positioning mold is in contact with the six optical fibers, with an application thickness of less than 2 mm, and then exposed to light with a wavelength of 450 nanometers for curing. After the curing, the optical fiber alignment plate 20 is removed upwards.

In some optional embodiments of the present disclosure, a separating agent may be applied to the surface of the optical fiber positioning mold before the application of the curing material, so as to reduce a resistance to removal after curing.

In step S7, a tubular portion 2 with an appropriate inner diameter is selected according to the number and diameter of the plurality of optical fibers 4, and the second ends of the optical fibers 4 left outside the optical fiber positioning mold are inserted into the tubular portion 2 so that the optical fibers 4 located between the tubular portion 2 and the optical fiber positioning mold form an umbrella-shaped portion. In an embodiment of the present disclosure, a stainless steel capillary tube with a length of 2 mm, an outer diameter of 1.8 mm and an inner diameter of 0.8 mm is selected, and the second ends of the plurality of optical fibers left outside the optical fiber positioning mold are inserted into the stainless steel capillary tube for about 3 mm.

In step S8, the reference optical fiber is inserted into the tubular portion 2 so that the optical fibers 4 are arranged surrounding the reference optical fiber in a pattern of concentric circle, square, rectangle or other shapes, with one end of the reference optical fiber being close to the optical fiber positioning mold. In an embodiment of the present disclosure, a seventh optical fiber is used as the reference optical fiber and inserted into the stainless steel capillary tube so that it is located at a center of the other six optical fibers. The seven optical fibers form concentric circles. The end of the seventh optical fiber inserted into the stainless steel capillary tube is close to the surface of the optical fiber positioning mold.

In step S9, a reflective paint is applied to an end face of the reference optical fiber close to the optical fiber positioning mold. In an embodiment of the present disclosure, a metallic paint is applied to the end face of the seventh optical fiber close to the optical fiber positioning mold.

In step S10, the tubular portion 2 with the optical fibers 4 inserted is pushed towards the optical fiber positioning mold to be as close to the mold as possible until it encounters a great resistance, so that the optical fibers 4 located between the tubular portion 2 and the optical fiber positioning mold form an umbrella-shaped portion.

In step S11, the umbrella-shaped portion and part of the tubular portion 2 are fixed using a curing material, so as to form a fixing portion 3. In an embodiment of the present disclosure, a black light-curing dental resin is applied to cover the umbrella-shaped portion outside the optical fiber positioning mold and part of the tubular portion 2, and then exposed to light with a wavelength of 450 nanometers for curing, so as to form a conical fixing portion 3.

In step S12, the cured optical fiber bundle cannula 14 is removed from the optical fiber positioning mold, and portions of the optical fibers 4 exposed outside the tubular portion 2 are cut off with a fiber cutter.

In some optional embodiments of the present disclosure, after the optical fiber bundle cannula 14 is cured, a light shielding paint may be applied to the surface of the optical fiber bundle cannula 14 to further enhance the light shielding property.

In step S13, the end surface of the tubular portion 2 is polished so that the end surfaces of the plurality of optical fibers 4 in the tubular portion 2 are smooth without scratches. In an embodiment of the present disclosure, the exposed end surface of the tubular portion 2 is polished using 1500-mesh, 3000-mesh, 7000-mesh, 10000-mesh and 12000-mesh sandpaper respectively.

In step S14, the optical fiber bundle cannula 14 is attached to the optical fiber connector 1, for example, the two may be fixedly attached using an adhesive.

If no reference optical fiber is provided, the above step 6 and step 7 may be omitted.

In such embodiments, the method of manufacturing the optical fiber bundle cannula is based on steps S1 to S14 described above. As shown in FIG. 6, in a usage scenario of six optical fibers, compared with a manufacturing method without using an optical fiber alignment plate, the manufacturing method using an optical fiber alignment plate may achieve a less variation of a fiber angle with respect to 90 degrees (angular deviations in two orthogonal directions are measured for each fiber, n=12) during a fiber insertion process. Therefore, a higher degree of alignment of the optical fiber is achieved. Upon the one-way ANOVA analysis, a significance test result P-value is less than 0.01.

According to the method of manufacturing the optical fiber bundle cannula and the multi-channel fiber photometry system of the present disclosure, it is possible to freely set the relative position of the plurality of optical fibers in the optical fiber bundle cannula and simultaneously target different brain regions, thereby improving the flexibility of recording different brain regions. The optical fiber bundle is tightly arranged and highly integrated, thus has a small size and a light weight, and may be carried around by an animal containing the targets to be measured. A commutating device is used to avoid a signal distortion caused by a cable deformation, and also avoid the problem of cable entanglement caused by activities of the animal containing the targets to be measured, so that a continuous recording may be performed for hours or even days.

It should also be noted that the directional terms mentioned in the embodiments, such as “upper”, “lower”, “front”, “back”, “left”, “right”, etc., are just for reference to the directions in the accompanying drawings, not intended to limit the scope of the present disclosure. Throughout the accompanying drawings, the same elements are denoted by the same or similar reference numerals. Conventional structures or constructions will be omitted when they are possible to cause confusions in the understanding of the present disclosure. Moreover, the shapes and sizes of the components in the accompanying drawings do not reflect actual sizes and scales, but only illustrate contents of the embodiments of the present disclosure.

The ordinal numbers used in the specification and claims, such as “first”, “second”, “third”, etc., to modify the corresponding elements do not mean that the elements have any ordinal numbers, nor do they represent the order of one element and another element, or the order in the manufacturing method. These ordinal numbers are just used to clearly distinguish an element with a particular name from another element with the same name.

The aforementioned specific embodiments further describe the purpose, technical solutions and beneficial effects of the present disclosure in detail. It should be understood that the above are just specific embodiments of the present disclosure and are not used to limit the present disclosure. Any modifications, equivalent substitutions, improvements, etc. made within the spirit and principles of the present disclosure should be contained in the protection scope of the present disclosure.

1. A method of manufacturing an optical fiber bundle cannula, comprising:
manufacturing an optical fiber positioning mold and forming a plurality of positioning holes on the optical fiber positioning mold;
inserting first ends of a plurality of optical fibers into the positioning holes of the optical fiber positioning mold respectively;
fixing exposed portions of the plurality of optical fibers close to the optical fiber positioning mold to the optical fiber positioning mold by using a curing material so that a relative position of ends of the plurality of optical fibers inserted into the optical fiber positioning mold remains unchanged;
inserting second ends of the plurality of optical fibers into a tubular portion so that the optical fibers located between the tubular portion and the optical fiber positioning mold form an umbrella-shaped portion;
fixing the umbrella-shaped portion and part of the tubular portion by using a curing material, so as to form a fixing portion; and
extracting the optical fibers inserted into the optical fiber positioning mold from the optical fiber positioning mold.

2. The method of manufacturing the optical fiber bundle cannula according to claim 1, wherein the inserting first ends of a plurality of optical fibers into the positioning holes of the optical fiber positioning mold respectively comprises:
manufacturing an optical fiber alignment plate, wherein the optical fiber alignment plate is provided with a plurality of alignment holes configured for the optical fibers to pass through, wherein the alignment holes are consistent with the positioning holes on the optical fiber positioning mold in terms of position and number;
placing the optical fiber alignment plate in parallel above the optical fiber positioning mold so that each of the alignment holes corresponds to a position of one of the positioning holes in a direction of an orthographic projection of the optical fiber positioning mold;
passing the first ends of the plurality of optical fibers respectively through corresponding alignment holes, so as to pre-position the optical fibers; and
inserting the first end of each optical fiber into the positioning hole;
wherein the alignment hole is a through hole.

3. The method of manufacturing the optical fiber bundle cannula according to claim 2, further comprising: after fixing the exposed portions of the plurality of optical fibers close to the optical fiber positioning mold to the optical fiber positioning mold by using the curing material so that the relative position of the ends of the plurality of optical fibers inserted into the optical fiber positioning mold remains unchanged,
removing the optical fiber alignment plate.

4. The method of manufacturing the optical fiber bundle cannula according to claim 1, wherein the positioning hole formed on the optical fiber positioning mold comprises a blind hole or a variable diameter hole.

5. The method of manufacturing the optical fiber bundle cannula according to claim 1, further comprising: after inserting the second ends of the plurality of optical fibers into the tubular portion,
inserting one or more reference optical fibers into the plurality of optical fibers so that the plurality of optical fibers are closely arranged around the reference optical fiber in a pattern of concentric circle, square, rectangle or other shape, and one end of the reference optical fiber is close to the optical fiber positioning mold.

6. The method of manufacturing the optical fiber bundle cannula according to claim 1, wherein the curing material has a light shielding property.

7. The method of manufacturing the optical fiber bundle cannula according to claim 1, further comprising: after extracting the optical fibers inserted into the optical fiber positioning mold from the optical fiber positioning mold,
cutting off portions of the plurality of optical fibers exposed from the tubular portion.

8. A multi-channel fiber photometry system, comprising:
a tubular portion, a fixing portion, and a plurality of optical fibers, wherein middle portions of the plurality of optical fibers are fixed in the fixing portion, and the first ends of the optical fibers are exposed from the fixing portion in order to be inserted into targets to be measured so as to conduct excitation light to the targets to be measured and collect emission light which is generated by the targets to be measured upon the targets to be measured is excited; and second ends of the plurality of optical fibers are held in the tubular portion; and
an optical fiber detection device attached to the optical fiber bundle cannula, and configured to generate the excitation light, receive the emission light, and convert an optical signal into an electrical signal.

9. The multi-channel fiber photometry system according to claim 8, wherein the optical fiber detection device comprises:
an optical fiber connector, wherein a tubular portion of the optical fiber bundle cannula is partially inserted into one end of the optical fiber connector to achieve an optical coupling between the optical fiber connector and the plurality of optical fibers of the optical fiber bundle cannula;
an optical transceiver configured to generate the excitation light and receive the emission light; and
an image sensor configured to generate an electrical signal representing an image of an object to be imaged, according to the emission light received by the optical transceiver.

10. The multi-channel fiber photometry system according to claim 9, wherein the optical transceiver comprises:
a housing having an interface optically coupled to the optical fiber connector;
a light source provided in the housing and configured to generate a light beam;
a first optical filter provided in the housing and configured to filter the light beam from the light source to generate the excitation light; and
an optical conversion assembly provided in the housing and configured to guide the excitation light from the first optical filter to the optical fiber bundle cannula and guide the emission light from the optical fiber bundle cannula to the image sensor.

11. The multi-channel fiber photometry system according to claim 10, wherein the optical conversion assembly comprises:
a dichroic mirror;
an objective lens provided between the dichroic mirror and the optical fiber connector and configured to receive the excitation light which is generated from the first optical filter and reflected by the dichroic mirror and the emission light from the optical fiber bundle cannula, and further inject the excitation light onto the optical fiber bundle cannula;
an eyepiece configured to receive the emission light which comes from the objective lens and is transmitted by the dichroic mirror; and
a second optical filter configured to filter the emission light from the eyepiece and guide filtered emission light to the image sensor.

12. The multi-channel fiber photometry system according to claim 8, wherein the second end of the multiple optical fibers form a plane.

13. The multi-channel fiber photometry system according to claim 8, further comprising:
a signal acquisition device configured to receive the electrical signal from the optical fiber detection device; and
a commutating device coupled between the optical fiber detection device and the signal acquisition device to avoid a cable entanglement caused by a movement of an animal that contains the targets to be measured.

14. The multi-channel fiber photometry system according to claim 9, further comprising:
a signal acquisition device configured to receive the electrical signal from the optical fiber detection device; and
a commutating device coupled between the optical fiber detection device and the signal acquisition device to avoid a cable entanglement caused by a movement of an animal that contains the targets to be measured.

15. The multi-channel fiber photometry system according to claim 10, further comprising:
a signal acquisition device configured to receive the electrical signal from the optical fiber detection device; and
a commutating device coupled between the optical fiber detection device and the signal acquisition device to avoid a cable entanglement caused by a movement of an animal that contains the targets to be measured.

16. The multi-channel fiber photometry system according to claim 11, further comprising:
a signal acquisition device configured to receive the electrical signal from the optical fiber detection device; and
a commutating device coupled between the optical fiber detection device and the signal acquisition device to avoid a cable entanglement caused by a movement of an animal that contains the targets to be measured.
